(19) United States
(12) Patent Application Publication
(10) Pub. No.: US 2015/0198589 A1
(43) Pub. Date: Jul. 16, 2015

(54) USE OF EDTA TUBE WITH GEL IN ELISA METHOD

(71) Applicants: Ekrem ERBIZ, (US); Tamer TOPBAS, (US)

(72) Inventors: Ekrem Erbiz, Istanbul (TR); Tamer Topbas, Istanbul (TR)

(21) Appl. No.: 14/407,945

(22) PCT Filed: May 24, 2013
(86) PCT No.: PCT/TR2013/000172

§ 371 Date: Dec. 13, 2014

(30) Foreign Application Priority Data
Jun. 15, 2012 (TR) 2012/07034

(51) Int. Cl. G01N 33/53

(52) U.S. Cl. CPC G01N 33/5306

(57) ABSTRACT

The invention relates to elimination of the false negative or defective results obtained as a result of inadequate or no sample collection by the test sampling probes in the analyzers running with full or semi-automatic Elisa method in the studies conducted from the serum today in determination of the anti HIV, anti HCV, Syphilis antibodies and HBSAg antigen by employing ELISA (Enzyme Linked Immune Sorbent Assay) method, one of the serologic methods, in full or semi-automatic analyzers by use of the plasma obtained from EDTA gel tube.

The invention relates to elimination of the false negative or defective results obtained as a result of inadequate or no sample collection by the test sampling probes in the analyzers running with full or semi-automatic Elisa method in the studies conducted from the serum today in determination of the anti HIV, anti HCV, Syphilis antibodies and HBSAg antigen by employing ELISA (Enzyme Linked Immune Sorbent Assay) method, one of the serologic methods, in full or semi-automatic analyzers by use of the plasma obtained from EDTA gel tube.

### State of the Art

The term ELISA is the abbreviation for “Enzyme Linked Immuno Sorbent Assay” test. It is a quantitative measurement method based on examining the antigen-antibody relation and the activity of an antibody-bound enzyme.

In the method mentioned above, it is possible to search the antibody against the antigen or the antigen against the antibody wherein said method is also a diagnosis method used for the virus and parasite infections. Here, non-competitive indirect staining method is employed by the use of the immobilized antigen.

ELISA method was discovered in 1960s as an alternative to “Radioimmunoassay” methods and the use thereof has spread wideworld ever since.

Due to the longevity of the reagents employed in ELISA method and the absence of a radiation hazard associated with the waste materials, ELISA rapidly become the method preferred over RIA (Radioimmunoassay) method.

One of the most important advantages of ELISA method is that it provides the ability to work with a great number of samples within a short time in the diagnosis laboratories. Upon the productions becoming widespread parallel with increasing usability of these tests, there was a reduction in their costs. For this reason, it is frequently used in many hospitals and laboratories, since it yields reliable and economical results.

From past to present, Macro and Micro ELISA assays have been performed from the “Serum”, the blood liquid, in Turkey and numerous countries of the world.

Serum is the name given to the light colored yellowish liquid that remains when the fibrinogen and platelets combine in the form of a dark colored mass of clot once the blood has lost its homogeneous structure after it has been kept to enable the clot formation. (In practice, in ELISA studies, this clotted portion and the shaped blood components remain in the bottom of the tube, while the fibrin clot remains within the serum.)

The blood clots a certain period of time after it is taken from the body and placed into a glass container. This condition results from the conversion of the plasma protein, which is present in dissolved state within the blood and which is referred to as fibrinogen, into the insoluble fibrin. The cells in the blood remain within this fibrin. A light colored yellowish liquid emerges as a result of the contraction of the fibrin and this liquid is called the serum.

The liquid obtained by the separation of the cell members from the unclotted blood is called the plasma. Whereas serum does not contain fibrinogen and some other coagulation factors, said substances are present within the plasma.

The antibody search in the ELISA method is performed in several ways. These are as follows:
Known antigen is adhered to a plastic surface. For Micro-Elisa system, this antigen is coated on the surface of the wells made for being used for each patient. The patient serum where the antibody is to be sought is placed into these wells. A certain period of time is allowed to elapse and washing is performed. In case the corresponding antibody is present in the serum, it combines with the antigen.
Human globulin antiserum marked with an enzyme is added. A certain period of time is allowed to elapse and washing is performed. In case the serum under examination contains the antibody that matches the antigen, it will become bonded to the antigen, hence it will bind also this last added enzyme-marked human antiglobulin and it will not be possible to be removed by way of washing.
A suitable chromogenic substrate is added to the enzyme. The color that appears when the system-bound enzyme breaks down this substrate will be measured by means of colorimetric methods and will give an idea about the bound enzyme and hence the bound antibody.

Today, anti HIV, anti HCV, HBSAg and Syphilis studies conducted with the ELISA method in full and semi-automatic analyzers in Turkey and in various countries of the world use blood fluid, i.e. the “serum”, which also contains fibrin clots therein; therefore, many disadvantages emerge from such use.

That is to say;

Fibrin present in the serum does not precipitate adequately in some persons after centrifuge process, or fibrin clot occurs in blood samples due to biochemical properties of the patient/donor. This ratio is observed in healthy individuals at 10-15% frequency.

Such clots cause occlusions in the probes of the auto-analyzer and full and semi-automatic ELISA analyzers during the study, or failure of the reagent to suck (pipetting) serum in quantities set forth in the instructions for use.

Pipetting from the serum process is performed as follows;
The blood samples collected from the patients and the donors are placed inside test tubes free of anticoagulant substance; then the serum is obtained through centrifuging at 4000 rpm for approximately 15 minutes.
The test samples to be studied and the reagents and consumables to be used during the tests are placed to the full and semi-automatic ELISA analyzer connected to the PC unit.
For the test study, serum is placed in the auto-analyzer and the system is turned on.
For pipetting process, the probe comes over the tube through horizontal and vertical motions and enters into the tube and sucks specified amount of serum from the tube.
Then, the probe leaves a suitable amount of serum to the test working environment, the inner surfaces of which are coated with antigen.
The probe encounters the fibrin block having same color as the serum and, if the software installed on the PC acknowledges that the pipetting (sucking desired amount of serum) process is completed, the probe drops serum into the working environment corresponding to the amount missing.
If the software installed on the PC considers that the pipetting process could not be performed (in cases where test sampling probes collect inadequate samples or collects no samples at all), then the probe does not release serum to the working environment. In this case, the analyzer continues the test processes unless warned by the user.

Failure of the analyzer to pipette specified quantity of serum (test sampling probes collect inadequate samples or collects no samples at all) not only affects the test results, but also causes loss of reagent, material and time.

If the control mechanism of the analyzer is not sensitive enough to detect the fact that the probes of the auto-analyzers pipetting the test sample pipette less amount of serum than the required test amount set forth in the instructions of the reagent, the auto-analyzer continues to operate.

If the analyzer receives serum samples less than the required test amount as set forth in the instructions of the reagent, the analyzer can cause defective test results. The analyzer might provide a positive sample as negative. This fact is an unacceptable and irredeemable situation especially for the blood centers.

The method commonly employed today for solving this problem is a solution method that is based on visual observation, which is completely dependent on the technician (people).

Until this day, aforementioned problems have been considered to be a drawback of the full and semi-automatic ELISA analyzers; and the operating system, safety software and mechanical parts of the analyzers have been developed continuously in order to eliminate said problem. The problem, however, could not be avoided despite all such investments.

Our invention is a practice which eliminates all the aforementioned disadvantages and defective measurements emerging accordingly, and which enables pipetting of required amount of samples (serum sampling at adequate amount by the test sample probes) and defect-free measurements/testing, wherein it is aimed to use EDTA gel tube, which has never been used during the scan tests worked with ELISA method, instead of the dry gel tube, where the serum, which is the test material used until this day, is obtained, in anti HIV, anti HCV, HBSAg and Syphilis blood analyses conducted in full and semi-automatic analyzers by employing micro and macro ELISA method, and thus to obtain the sample as plasma.

The term EDTA is the abbreviation for ethylene diamine tetra acetic acid. EDTA is a polyamino carboxylic acid compound. EDTA was first described by Ferdinand Munz. Munz achieved the discovery of EDTA from ethylenediamine and chloroacetic acid solutions.

Since the EDTA tubes with gel contain anticoagulant agent, no fibrin clot forms from fibrinogen within the tube during the procedure. Our invention enables to avoid the erroneous measurements and detections in the serums with fibrin clot as well as enabling an error-free detection and measurement. Since the existing EDTA tubes do not have a gel barrier, the shaped blood components may become broken down in time, leading to haemolysis, which in turn deteriorates the plasma quality.

Moreover, the required centrifuge period and revolution are lower for obtaining plasma from EDTA gel tubes when compared to the serum tubes. While it suffices to apply centrifuge process at 3000 rpm for 5-10 minutes for obtaining plasma; serum requires a centrifuge process of minimum 15 minutes at 4000-5000 rpm. This, in turn, increases the risk of hemolysis in the blood depending on the centrifuge period and rotation speed and affects the quality of the study material.

As mentioned above, our invention eliminates the erroneous or defective sampling in ELISA assays.

Another characteristic of our invention is that the loss of used material is eliminated. No loss of material leads to savings on time and effort. The rate of efficiency in anti HIV, anti HCV, HBSAg and Syphilis studies conducted by employing ELISA method in full and semi-automatic analyzers reach to 99.99% at EDTA gel tubes.

Our invention enables precise test results as the study is conducted on the samples at quantities set forth in the instructions for use obtained through pipetting process depending on chitin sensitivity.

Another characteristic of our invention is that, if disposable pipettes are not used during the study (in auto-analyzers with Teflon probes, etc.), the risk of contamination is prevented. When Teflon coated pipettes get in contact with the fibrin clot during the studies conducted with the serum, they might contaminate the next sample as the fibrin contaminate cannot be cleaned completely, thus have negative impact on the efficiency due to contamination.

### DESCRIPTION OF THE INVENTION

Our invention is intended for obtaining defect-free test results by preventing defective sample pipetting from the serum by the probes performing pipetting operation for the auto-analyzer through use of EDTA gel tubes in the scan tests conducted with ELISA method in anti HIV, anti HCV, HBSAg and Syphilis blood analyses performed in full and semi-automatic analyzers by employing micro and macro ELISA method.

Our invention relates to execution of the anti HIV, anti HCV, HBSAg and Syphilis blood tests performed in full and semi-automatic analyzers by employing micro and macro ELISA method using EDTA gel tube in the analyzers.

In our invention, the analysis result is obtained from plasma as said anti HIV, anti HCV, HBSAg and Syphilis blood tests performed in micro and macro auto-analyzers by employing ELISA method are now performed with EDTA gel tube during the scan tests worked with ELISA method.

EDTA (Ethylene Diamine Tetra Acetic Acid) is an anticoagulant agent. Since the assay sample in the tube is obtained as plasma after the centrifugation in the machine/device, it does not contain fibrin clot.

EDTA (ethylene diamine tetra acetic acid) is an anticoagulant substance. As the work sample at the EDTA gel tube content is obtained as plasma, the work sample does not contain fibrin clot due to its structure. This, in turn, eliminates the risk of encountering fibrin block during operation of the probes at full and semi-automatic analyzer where ETDA gel tube is used, thus eliminating the defective measurements.

As the tube developed for use in full and semi-automatic analyzers in our invention contains gels, after the centrifuge (circular rotational motion, application of centrifugal force, centrifugal moment) process applied in the analyzers, shaped elements remain under the gel and do not mix with the plasma. As fibrin formation, mixing of shaped elements to the plasma and deterioration of the plasma quality due to hemolysis at the work sample are avoided in this manner, factors that will block the probe of the auto-analyzer and the factors that will affect the test results are minimized at the work sample. This, in turn, leads to obtaining reliable test results and is more economic as there will be no loss of material and kits.

As the anti HIV, anti HCV, HBSAg and Syphilis blood analyses conducted in full and semi-automatic analyzers by employing micro and macro ELISA method are now performed with EDTA gel tube, which has never been used at the scan tests employing ELISA method, in our invention, the effects of the defects at the technical equipment of the probes executing pipetting (serum sampling at desired rate) process in full and semi-automatic analyzers running on ELISA method, wherein the tube is positioned, on the reliability of the study are minimized.

As the anti HIV, anti HCV, HBSAg and Syphilis blood analyses conducted at the full and semi-automatic analyzers by employing micro and macro ELISA method are performed with EDTA gel tube, which has never been used at the scan tests employing ELISA method, in our invention, loss of materials and kits is completely eliminated, thus economic savings are achieved on national and firm basis.

1. Tubes used in full and semi-automatic analyzers (auto-analyzer) where the blood is placed for conducting anti HIV, anti HCV, HBSAg and Syphilis blood tests by employing micro and macro ELISA method, and the analyzers (auto-analyzer) where said tubes are used, characterized in that the anti HIV, anti HCV, HBSAg and Syphilis blood analyses by employing micro and macro ELISA method are performed in full and semi-automatic analyzers (auto-analyzer) by using EDTA (ethylene diamine tetra acetic acid) gel tube in order to prevent defective test results arising from the tube of the analyzers (auto-analyzer).

2. Tubes used in full and semi-automatic analyzers (auto-analyzer) wherein the blood is placed for conducting anti HIV, anti HCV, HBSAg and Syphilis blood tests by employing micro and macro ELISA method, characterized in that the tubes used in full and semi-automatic analyzers (auto-analyzer) for conducting anti HIV, anti HCV, HBSAg and Syphilis blood tests are EDTA (ethylene diamine tetra acetic acid) gel tubes.

3. Analyzers (auto-analyzer) containing the tubes in which the blood is placed for conducting anti HIV, anti HCV, HBSAg and Syphilis blood tests by employing micro and macro ELISA method, and testing the tubes, characterized in that the tubes used in full and semi-automatic analyzers (auto-analyzer) for conducting anti HIV, anti HCV, HBSAg and Syphilis blood tests contain EDTA (ethylene diamine tetra acetic acid) gel.

4. Analyzers (auto-analyzer) with probes of Teflon, etc. conducting anti HIV, anti HCV, HBSAg and Syphilis blood tests by employing micro and macro ELISA method, characterized in that the tubes used for preventing defective sampling and contamination in anti HIV, anti HCV, HBSAg and Syphilis blood tests conducted in full and semi-automatic analyzers with fixed Teflon etc. probes (auto-analyzer) contain EDTA (ethylene diamine tetra acetic acid) gel.
